(19) United States
(12) Patent Application Publication
(10) Pub. No.: US 2009/0098088 A1
(43) Pub. Date: Apr. 16, 2009

(54) Methods And Kits For The Treatment Of Diverticular Conditions

(75) Inventors: Kevin Douglas Taylor, Morrow, OH (US); Simon Henry Magowan, Loveland, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(73) Assignee: The Procter & Gamble Company, Cincinnati, OH (US)

(21) Appl. No.: 12/246,699

(22) Filed: Oct. 7, 2008

Related U.S. Application Data
(60) Provisional application No. 60/998,403, filed on Oct. 10, 2007.

(51) Int. Cl. A61K 35/74; A61P 1/00

(52) U.S. Cl. 424/93.4

(57) ABSTRACT

The present invention relates to methods and kits that are useful for the treatment of diverticular disease, diverticulitis, and combinations thereof. The methods comprise administering to a mammal in need of treatment a composition comprising a probiotic, either alone or in combination with an anti-inflammatory or an antibiotic.

### CROSS REFERENCE TO RELATED APPLICATIONS

This application claims the benefit of U.S. Provisional Application No. 60/998,403 filed on Oct. 10, 2007.

### FIELD OF THE INVENTION

The present invention is directed to methods and kits that are useful for the treatment of diverticular disease, diverticulitis, and combinations thereof. The methods comprise administering to a mammal in need of treatment a composition comprising a probiotic, either alone or in combination with an anti-inflammatory or an antibiotic.

### BACKGROUND OF THE INVENTION

Diverticular disease and diverticulitis are gastronintestinal conditions estimated to affect over two million people per year in the United States. Diverticular conditions may be the most common structural abnormalities of the colon in Europe and North America, and increase with advancing age.^{1,2,3} These conditions most commonly involve the large colon. Typically, following an attack of diverticulitis, abdominal symptoms will persist in 65% of patients after the first attack and in 90% of patients after the second attack.⁴

Diverticular conditions of the colon are the fifth most common important gastrointestinal conditions in terms of direct and indirect healthcare costs.⁵ There are approximately 450,000 inpatient hospital stays for diverticular conditions in the United States annually. For perspective, ulcerative colitis (UC), which has FDA-approved treatments, is responsible for approximately 46,000 hospital admissions per year in the US.⁶

It is believed that no disease-modifying pharmacologic intervention is currently approved by the regulatory authorities in North America or Europe to prevent acute diverticular attacks. Copious literature exists for diverticular conditions on the impact of CT imaging and the timing of surgical intervenion.^{9,10,11,12,13,14,15,16,17,18,19} Little research exists regarding the natural history of acute diverticulitis, the time course of inflammation and clinical sequelae, and whether this can be lessened by pharmacologic intervention.^{20,21,22} It is unknown whether chronic colonic inflammation causes persistent gastrointestinal symptoms following an acute attack or is predictive for further diverticular attacks.²³

The efficacy of treatment recommendations offered to patients after an acute attack of diverticulitis is inadequate. Increased dietary fiber is a recommended treatment for the patient after an acute attack of diverticulitis. A recommended dietary fiber intake for adults is 20-35 g/day, but the average intake in the West is only 14-15 g/day.²⁴ Epidemiological evidence suggests that not only would such treatment need to be prolonged, but also the amount of fiber needed would be very high, much higher than the typical Western “high fiber diet.”^{25,26} As the population ages, the incidence of diverticular conditions will continue to increase, and primary prevention with a high fiber diet is probably not a feasible management option.²⁷

A therapeutic manipulation of the colonic flora may decrease colonic inflammation and ameliorate symptoms.

### SUMMARY OF THE INVENTION

The present invention is directed to methods and kits for the treatment of diverticular conditions. In one embodiment, the invention is directed to a method of treating a condition selected from diverticular disease, diverticulitis, and combinations thereof, the method comprising administering to a mammal in need of such treatment a composition comprising a Bifidobacterium spp. In another embodiment, the invention is directed to a method of treating a condition selected from diverticular disease, diverticulitis, and combinations thereof, the method comprising administering to a mammal in need of such treatment a probiotic, an anti-inflammatory, and an antibiotic.

In yet another embodiment, the invention is directed to kits, wherein the kits comprise a probiotic, which may be a Bifidobacterium spp. but need not be, and an anti-inflammatory. The kits may optionally comprise an antibiotic.

### BRIEF DESCRIPTION OF THE DRAWINGS

FIG. 1 is a Diagram and shows a description of the 3 treatment regimens for 12 weeks standard of care patients receive;

FIG. 2 is a Table and shows a description of the schedule of events; and

FIG. 3 is a Chart and shows a description of the Gastrointestinal symptom severity assessment based on the patients' recall of the previous 3 days, using the Global Symptom Score (GSS) tool.

### DETAILED DESCRIPTION OF THE INVENTION

All documents cited herein are, in relevant part, incorporated herein by reference; the citation of any document is not to be construed as an admission that it is prior art with respect to the present invention.

Except where specific examples of actual measured values are presented, numerical values referred to herein should be considered to be qualified by the word “about”.

### Methods of the Present Invention

The present invention is directed to methods and kits for the treatment of diverticular conditions. In one embodiment, the invention is directed to a method of treating a condition selected from diverticular disease, diverticulitis, and combinations thereof, the method comprising administering to a mammal in need of such treatment a composition comprising a Bifidobacterium spp. In another embodiment, the invention is directed to a method of treating a condition selected from diverticular disease, diverticulitis, and combinations thereof, the method comprising administering to a mammal in need of such treatment a probiotic, an anti-inflammatory, and an antibiotic.

Diverticular conditions may be associated with a variety of symptoms including, for example, abdominal pain, abdominal tenderness, nausea, vomiting, bloating, constipation, diarrhea, mucus in stool, feeling urge to evacuate but no bowel movement, painful straining with bowel movement, and pain or difficulty urinating and, as such, the methods herein encompass treatment of any of these variety of symptoms.

As used herein, the term “administration”, “administering”, “or the like with respect to the user means that the user is administered, is directed to administer or, with reference specifically to “oral administration,” or “orally administering,” ingests or is directed to ingest, the composition. For example, the administration may be oral administration, parenteral administration, topical administration, buccal administration, rectal administration, or the like, or any combination thereof. As but one example, the anti-inflammatory may be administered orally or rectally, while the probiotic may be administered orally. In one embodiment, all components are administered through oral administration.

Wherein the user is directed to administer the composition or component, such direction may be that which instructs and/or informs the user that use of the composition or component (as applicable) may and/or will provide one or more general health and/or general physiological benefits associated with the health care product. For example, such direction may be oral direction (e.g., through oral instruction from, for example, a physician, health professional, sales professional or organization, and/or radio or television media (e.g., advertisement) or written direction (e.g., through written direction from, for example, a physician or other health professional (e.g., scripts), sales professional or organization (e.g., through, for example, marketing brochures, pamphlets, or other instructive paraphernalia), written media (e.g., internet, electronic mail, or other computer-related media), and/or containing devices associated with the composition (e.g., a label present on a containing device containing the composition). See e.g., the kits described herein.

### The Probiotic

The methods of the present invention utilize a probiotic. Without being bound by theory, it is believed that the present invention has utility based on an immunological and microbiological stance. Probiotics have been shown to inhibit pathogen adherence to colonic mucosa, increase immunoglobulin-A (IgA) secretion in Peyer's patches, increase immune activity inhibiting the release of anti-inflammatory cytokines and inhibiting pro-inflammatory cytokines ². Probiotics may also interfere with pathogen metabolism.

Advancing age may be associated with decreased bifidobacteria and increased Bacteroides species, a major pathogen in acute diverticulitis attacks.^{29,30} Although it is now accepted that low fiber diets are associated with diverticular formation, it should also be noted that low fiber diets may actually alter the colonic microecology: it has been demonstrated in humans that wheat bran adversely changes the anerobic/aerobic bacterial ratios.³¹ This has implications for the application of probiotics as prophylaxis against bacteroides overgrowth and infection leading to acute diverticular conditions.

In the practice of the present invention, the probiotic may be, for example, lactic acid bacteria. Non-limiting examples of lactic acid bacteria suitable for use herein include strains of Streptococcus lactis, Streptococcus cremoris, Streptococcus diacetylactis, Streptococcus thermophilus, Lactobacillus bulgaricus, Lactobacillus acidophilus, Lactobacillus helveticus, Lactobacillus bifidus, Lactobacillus casei, Lactobacillus lactis, Lactobacillus plantarum, Lactobacillus rhamnosus, Lactobacillus delbruekii, Lactobacillus thermophilus, Lactobacillus fermentii, Lactobacillus salivarius, Bifidobacterium longum, Bifidobacterium infantis, Bifidobacterium bifidum, and Pediococcus cerevisiae, or mixtures thereof. In one embodiment the probiotic is Lactobacillus salivarius, Bifidobacterium infantis, or mixtures thereof. In another embodiment, the probiotic is Bifidobacterium infantis.

As a non-limiting example, strains of Lactobacillus salivarius isolated from resected and washed human gastrointestinal tract as described in WO 98/35014 are preferred. More preferred are the Lactobacillus salivarius strains that are designated UCC 1 and UCC 118, described as being deposited at the National Collections of Industrial and Marine Bacteria Ltd (NCIMB) on Nov. 27, 1996, and accorded the accession numbers NCIMB 40830 and 40829, respectively.

As another non-limiting example, the probiotic is a Bifidobacterium spp. For example, strains of Bifidobacterium spp. isolated from resected and washed human gastrointestinal tract as disclosed in WO 00/42168 may be used herein. One example is the Bifidobacterium infantis strain designated as UCC35624, described as being deposited at the National Collections of Industrial and Marine Bacteria Ltd (NCIMB) on Jan. 13, 1999, and accorded the accession number NCIMB 41003.

The compositions used herein may be given to an individual as part of a dose regimen which may be dependent upon the dosing format used in which the probiotic is incorporated. For example, the unit dose provides the mammal being treated with probiotic at a level of from about 1×10⁵ colony forming units (cfu) per dose to about 1×10¹⁵ cfu per dose, alternatively from about 1×10⁷ cfu to about 1×10¹⁴ cfu per dose. As another example, the dose regimen may commence at a higher dose, followed by a lower maintenance dose. See, for example, U.S. Provisional Ser. No. 60/920,177, filed Mar. 20, 2007.

Any of a variety of different dose forms may be appropriate for administration. For example, for oral administration, the unit dose, when provided as a capsule, tablet, or other typical oral dosage form may be swallowed directly. As another example, when provided as a sachet filled with the probiotic, the probiotic may be ingested directly, or mixed with milk, yoghurt, or another liquid carrier material. Typically, as an example, a dose form such as a capsules may provide lower dosing amounts than sachets, as the size of the capsule, and its relative easy of ingestion, will limit the amount of the probiotic that can be filled therein.

### The Anti-Inflammatory

In certain embodiments, the methods of the invention comprise administration of an anti-inflammatory. Anti-inflammatories are commonly known and are selected by one of ordinary skill in the art. As examples, the anti-inflammatory may include those selected from salicylates, arylalkanoic acids, 2-arylpropionic acids, N-arylanthranillic acids, pyrazolidines, oxicams, coxibs, sulphonanilides, licofelone, omega fatty acids, and combinations thereof.

In one embodiment herein, the salicylate may be an aminosalicylate. As used herein, “aminosalicylate” refers to a class of compounds capable of releasing 5-amino-2-hydroxybenzoate or 5-amino-2-hydroxybenzoic acid as an active moiety in vivo. Non-limiting examples include mesalamine (5-amino-2-hydroxybenzoic acid), olsalazine (3,3′-dicarboxy-4,4′-dihydroxyazobenzene), balsalazide ((E)-5-[[4-[[(2-carboxyethyl)amino]carbonyl]phenyl]azo]-2-hydroxybenzoic acid), and sulfasalazine (2-hydroxy-5-[[4-[(2-pyridinylamino)sulfonyl]phenyl]azo]-benzoic acid).

A composition comprising an aminosalicylate may have one or greater than one aminosalicylate in addition to other possible components. The active moiety is illustrated below:

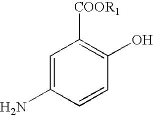

wherein R₁ can be hydrogen or a physiologically relevant counterion and the nitrogen can be further protonated and carry a positive charge along with a physiologically relevant counterion.

Although the examples provided describe the free acid or free amine forms, the term is not so limited and should be interpreted to include the free acid forms, the free amine forms, and any salts thereof. For example, the term “mesalamine” covers the free acid, the free amine, and any salts of mesalamine. The term “mesalamine” is commonly used interchangeably in the art with “mesalazine”, “5-ASA” or “5-aminosalicylic acid”.

In one embodiment herein, the anti-inflammatory is formulated for release in the small intestinal and/or the large intestine, such as for example at the distal portion of the small intestine and in the large intestine. Various illustrative commercial products are suitable for such release including, for example, ASACOL, PENTASA, and LIALDA. Various delayed or sustained delivery technologies are well known for this purpose and need not be described herein.

In accordance with the methods herein, as an example, from about 50 mg to about 8000 mg of the anti-inflammatory may be administered, or from about 100 mg to about 6000 mg, or from about 200 mg to about 4800 mg, or from about 1000 mg to about 4800 mg, or from about 1500 to about 4800 mg or from about 2400 mg to about 4800 mg. These doses are typically daily doses, although the ordinarily skilled artisan may manipulate dosing as needed or desired. For example, wherein the anti-inflammatory is available in discrete compositions each comprising 400 mg of the anti-inflammatory, a daily dose of 2400 mg may be administered through administration of six of these discrete compositions per day.

In certain embodiments herein, the anti-inflammatory is administered prior to administration of the probiotic. In this embodiment, the anti-inflammatory is administered for a definite period of time (for illustration, three times daily for a ten week period of time); upon conclusion of this illustrative ten week period of time, administration of the probiotic commences and continues for a definite period of time or, optionally, indefinitely.

In other embodiments herein, the anti-inflammatory is administered contemporaneously with administration of the probiotic. As used herein, “contemporaneously” means that, for any given day of administration, the anti-inflammatory and the probiotic are both administered on that day (whether at the same time, or at different times during that day).

In other embodiments herein, the anti-inflammatory is administered prior to administration of the probiotic and is also administered contemporaneously with the probiotic. To illustrate this embodiment, the anti-inflammatory is administered for a definite period of time (for illustration, three times daily for a ten week period of time); upon conclusion of this illustrative ten week period of time, administration of the anti-inflammatory continues along with commencement of administration of the probiotic, with anti-inflammatory and probiotic dosing for a definite period of time or, optionally, indefinitely.

### The Antibiotic

Current standard therapy using antibiotics alone may address acute attacks, but not thereafter the attack (e.g., symptoms may persist). As an example, in order to conform with the current standard of care, in certain embodiments, the methods may optionally comprise administration of an antibiotic.

Antibiotics are commonly known and are selected by one of ordinary skill in the art. To illustrate, the antibioitic may be selected from the group consisting of metronidazole, cephalexin, ciprofloxacin, levofloxacin, moxifloxacin, gemifloxacin, balofloxacin, gatifloxacin, grepafloxacin, pazufloxacin, sparfloxacin, temafloxacin, enoxacin, fleroxacin, lomefloxacin, nadifloxacin, norfloxacin, ofloxacin, pefloxacin, rufloxacin, trovofloxacin, tosufloxacin, clindamycin, tetracycline, chloramphenicol, cefoxitin, cefmetazole, cefotetan, doxycycline, erythromycin, imipenem, meropenem, ticarcillin, pipercillin, mezocillin, tazobactam, ampicillin, nitrofurantion, amoxicillin, trimethoprim, sulfamethoxazole, rifampin, penicillin, penicillin g, gentamicin, vancomycin, cefotaxime, ceftriazone, naldixic, cinoxacin, amifloxacin and combinations thereof.

As illustrative guidelines, subjects presenting with mild diverticular symptoms may be dosed a single type of antibiotic, while those presenting with more severe symptoms may be dosed a combination of antibiotics, including two, three, or even more distinct antiobiotics. For example, two distinct antibiotics may be prescribed for those with moderate symptoms, while triple therapy may be prescribed for those presenting with severe symptoms. In many of these moderate to severe cases, intravenous therapy may be appropriate, while typically oral dosing is also appropriate.

Dose level and frequency will be commonly understood in the art, and may be dependent upon the antibiotic employed. Merely as illustration, the methods and kits herein may optionally utilize a daily dose of from about 50 mg to about 6000 mg of antiobiotic, or from about 100 mg to about 2500 mg of antibiotic, or from about 250 mg to about 2000 mg of antibiotic. Daily dosing may be administered as a single dose, or divided into multiple doses such as twice daily, three times daily, or four times daily dosing.

To illustrate, the methods and kits herein may utilize administration of metronidazole (for example, FLAGYL). The metronidazole may optionally be administered orally in tablet form, optionally in immediate or sustained release forms. Other useful forms may include topical or intranvenous forms, or any other form that would be useful herein. A commonly used oral dose may include administration of from about 250 mg to about 750 mg of the metronidazole on a daily basis until antibiotic administration is complete.

As yet another illustration, the methods and kits herein may utilize administration of cephalexin (for example, KEFLEX, KEFTABS, or BIOCEF). The cephalexin may optionally be administered orally in tablet form, optionally in immediate or sustained release forms. Other useful forms may include powders for suspension, or any other form that would be useful herein. A commonly used oral dose may include administration of from about 250 mg to about 750 mg of the cephalexin on a daily basis until antibiotic administration is complete.

As yet another illustration, the methods and kits herein may utilize administration of doxycycline (for example, VIBRAMYCIN). The doxycycline may optionally be administered orally in tablet form, optionally in immediate or sustained release forms. Other useful forms may include suspensions, or any other form that would be useful herein. A commonly used oral dose may include administration of from about 50 mg to about 300 mg of the doxycycline on a daily basis until antibiotic administration is complete.

As yet another illustration, two or more discrete antibiotics may be utilized. For example, one may choose an antibiotic having anaerobic bacteria kill, and a different antibiotic having aerobic bacteria kill. For example, the methods and kits could utilize ciprofloxacin at a range of from about 250 mg per day to about 2000 mg per day along with metranidazole at a range of from about 250 mg per day to about 6000 mg per day.

In one embodiment, administration of the antibiotic is prior to the administration of the probiotic, prior to the administration of the anti-inflammatory, or prior to the administration of the probiotic and the anti-inflammatory. In certain embodiments, administration of the antibiotic is prior to the administration of the anti-inflammatory and the probiotic. For example, the antibiotic may be administered for a period of from 1 day to about 30 days following an acute attack of a diverticular condition, or from 1 day to about 14 days, or from about 7 days to about 10 days. The anti-inflammatory and the probiotic may also be administered, either contemporaneously or during different time periods.

The foregoing described dosage levels for anti-inflammatory, probiotic, and antibiotic are based on typical human subjects (e.g., about a 55 to 65 kg subject). Wherein the present composition is used in other mammals, it may be necessary to modify the dosage. Modification of dosage based on the needs of the subject is well within the skill of the ordinary artisan. It is therefore understood that these dosage ranges are by way of example only, and that administration can be adjusted depending on various factors.

The specific dosage of the anti-inflammatory, probiotic, and antibiotic, as well as the duration of treatment may be interdependent. The dosage and treatment regimen may also depend upon such factors as the specific anti-inflammatory, probiotic, and antibiotic used, as applicable, the treatment indication, the efficacy of the agent used, the personal attributes of the subject (such as, for example, weight, age, gender, and medical condition of the subject), and compliance with the treatment regimen.

### Kits of the Present Invention

In yet another embodiment, the invention is directed to kits, wherein the kits comprise a probiotic, which may be a Bifidobacterium spp. but need not be, and an anti-inflammatory. The kits may optionally comprise an antibiotic.

The probiotics, anti-inflammatories, and antibiotics, including various embodiments or selections thereof, are as described herein.

In one embodiment, the kits comprise one or more discrete compositions comprising the probiotic and one or more discrete compositions comprising the anti-inflammatory. For example, the kit may comprise a weekly, monthly, or other periodic dose of the probiotic and the anti-inflammatory. As an illustrative example, a kit comprising a weekly dose may comprise 7 discrete compositions comprising the probiotic (7 daily doses) and 42 discrete compositions comprising the anti-inflammatory (7 daily doses, each complete daily dose comprised of six discrete compositions). As another example, a kit comprising a monthly dose may comprise 30 discrete compositions comprising the probiotic (30 daily doses) and 180 discrete compositions comprising the anti-inflammatory. These kits may optionally comprise the antibiotic, for example in accordance with the number of doses of antibiotic intended for use. For example, wherein it is desired that 10 daily doses of antibiotic is administered prior to administration of the anti-inflammatory and prior to the probiotic, the kit may optionally contain 10 discrete compositions comprising the antibiotic. Any of a variety of combinations of types and numbers of discrete compositions will be selected by those of ordinary skill in the art.

In certain embodiments, the kits are configured to facilitate dosing compliance. For example, the kits may be particularly advantageous for the purpose of ensuring that the subject is receiving administration of all components (for example, probiotic, anti-inflammatory, and antibiotic) on the appropriately prescribed schedule. Blister cards or other containing devices appropriately configured may be particularly suitable for clearly illustrating sequence or timing of administration of the various components. Various configurations will be well known to the ordinarily skilled artisan in view of the present specification.

The kits of the present invention may comprise one or more of the probiotic, anti-inflammatory, and antibiotic, optionally together with information which informs a user of the kit, by words, pictures, and/or the like, that use of the kit will provide one or more general health and/or general physiological benefits including, but not limited to, gastrointestinal health benefits (for example relief from, prevention of, treatment of and/or inhibition of a diverticular condition), and/or general anti-inflammatory benefits. Such information need not utilize the actual words used herein, for example, “diverticular”, “diverticulitis”, “disease”, “condition”, or “gastrointestinal”, but rather use of words, pictures, symbols, and the like conveying the same or similar meaning are contemplated within the scope of this invention.

In one embodiment, the information is printed on a container holding the composition, e.g., a box, card (e.g., a blister card), or other containing device. These preferred kits may be in the form of one containing device containing the composition, or may be obtained as a plurality of devices each containing the composition. For example, the kits may be obtained as one card, or cases of four, six, seven (e.g., a weekly supply), or eight cards co-packaged together. Additionally, monthly or other types of kits may be obtained.

### Method of Manufacture

The compositions and various components used in the present invention may be manufactured in accordance with known methods.

### NON-LIMITING EXAMPLE

A study is placed to investigate the therapeutic effect of ASACOL® (a product containing mesalamine) in association with ALIGN® (a product containing the probiotic Bifidobacterium infantis 35624), in patients with diverticulitis. This example may be modified by those of ordinary skill in the art by substituting mesalamine with another anti-inflammatory and/or substituting Bifidobacterium infantis 35624 with another probiotic.

Scientific rigor is enhanced in this study compared to previous diverticulitis studies by the following features: double-blind placebo-controlled trial design; diagnosis of acute diverticulitis confirmed by computed axial tomography (CT); exclusion of irritable bowel syndrome (IBS) patients; constant daily dose of 5-ASA vs. variable cyclic dose regimens; and inclusion of serum, fecal, and time profiles for surrogate markers of inflammation.

The study is a 52-week, randomized, multicenter, double-blind, double-dummy, placebo-controlled, proof-of-concept (POC) study to evaluate the safety and efficacy of a 12-week treatment with ASACOL® (commercially available from The Procter & Gamble Company) followed by a 9-month non-treatment observation period in patients with acute diverticulitis. ASACOL® are administered with or without supplementation with ALIGN® (product containing Bifidobacterium infantis 35624, commercially available from The Procter & Gamble Company).

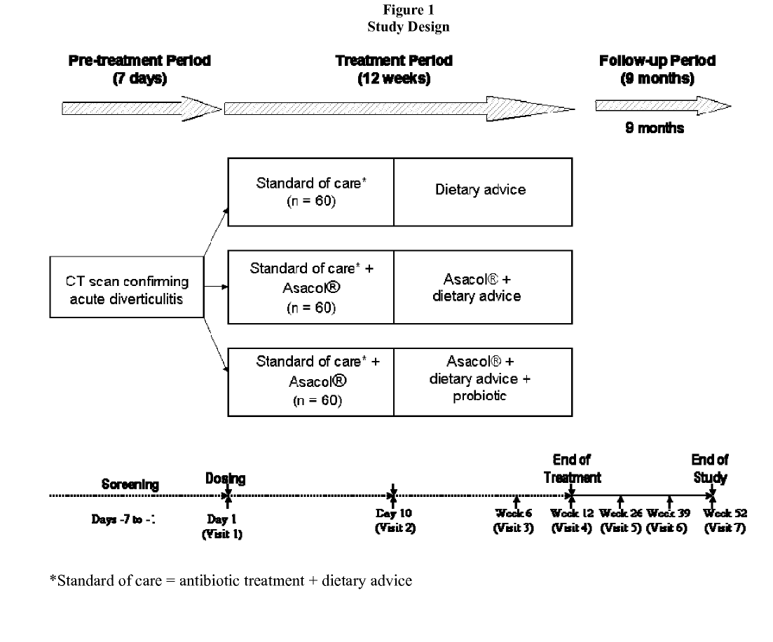

Patients are randomized to receive one of 3 treatment regimens for 12 weeks (FIG. 1):
Standard of care (which includes antibiotic for acute diverticulitis and dietary advice);
Standard of care (as defined above) and ASACOL®, started within 7 days of screening; or,
Standard of care (as defined above) and ASACOL® (started within 7 days of screening)+ supplementation with ALIGN®, which will start at Visit 2 (Day 10+4), after the patients have completed their antibiotic treatment regimen for acute diverticulitis.

ASACOL® and placebo to match Asacol or Align are manufactured by The Procter & Gamble Company and is packaged and labeled by Aptuit. ALIGN® capsules are made by JB Laboratories, Holland, Mich. Commercially packaged ALIGN® is obtained directly from the packager (Anderson Packaging, Rockford, Ill.).

### Asacol

Each ASACOL® delayed-release tablet for oral administration contains 400 mg of mesalamine, an anti-inflammatory drug. The 400 mg tablets are coated with acrylic-based resin coating, which is designed to release mesalamine in the terminal ileum and beyond for topical anti-inflammatory action in the colon.

ASACOL® is supplied as unprinted film-coated, red-brown, capsule-shaped tablets. Excipients include colloidal silicon dioxide, dibutyl phthalate, iron oxide red, iron oxide yellow, lactose, magnesium stearate, methacrylic acid copolymer B, polyethylene glycol, povidone, sodium starch glycolate, and talc.

### Placebo to Match Asacol

The placebo tablets resemble the active tablets in size and appearance and are supplied as unprinted film-coated, red-brown, capsule-shaped tablets. Excipients include ethyl cellulose, microcrystalline cellulose, lactose, dibutyl phthalate, sodium starch glycolate, iron oxide red, iron oxide yellow, povidone, talc, magnesium stearate, methacrylic acid copolymer B, and polyethylene glycol.

### Align

The Align® capsules are opaque white hydroxypropylmethylcellulose (non-gelatin, USP grade) capsules imprinted with “Align” and with a blue stripe (FD&C blue #2). The capsules contain Bifidobacterium infantis 35624, microcrystalline cellulose, magnesium stearate, sugar, sodium caseinate (milk protein), sodium citrate dehydrate, and propyl gallate.

### Placebo to Match Align

The placebo capsules to match Align use identical non-gelatin capsules containing only inert ingredients (microcrystalline cellulose, starch, and magnesium stearate).

### Placebo Group:

During the first 10 (+4) days, patients receive six placebo tablets once daily to match ASACOL® 400 mg in addition to “standard of care” (antibiotics + dietary advice). After antibiotic treatment has been completed, once daily placebo capsules to match ALIGN® are added to the regimen.

### Asacol-Only Group:

During the first 10 (+4) days, patients receive six 400 mg ASACOL® tablets once daily (six 400 mg ASACOL® tablets once daily) in addition to “standard of care” (antibiotics + dietary advice). After antibiotic treatment has been completed, once daily placebo capsules to match Align are added to the regimen.

### Asacol + Bifidobacterium Infantis 35624 (Probiotic) Group:

During the first 10 (+4) days, patients will receive 6×400 mg ASACOL® tablets once daily (six 400 mg ASACOL® tablets once daily) in addition to “standard of care” (antibiotics + dietary advice). After antibiotic treatment has been completed, once daily ALIGN® capsules are added to the regimen.

The 3 treatment regimens are summarized below:

| | | Route of |
|---|---|---|
| Treatment Group | Dose Regimen | Administration |
| Placebo | Placebo to match Asacol | Oral |
| | (6 tablets once daily; QD) | |
| | PLUS | |
| | Placebo to match Align ® | |
| | (1 capsule once daily, QD) | |
| ASACOL ®-Only | ASACOL ® (6 tablets × 400 mg | Oral |
| | once daily; QD) | |
| | PLUS | |
| | Placebo to match Align ® | |
| | (1 capsule once daily, QD) | |
| ASACOL ® + | ASACOL ® (6 tablets × 400 mg | Oral |
| Bifidobacterium | once daily; QD) | |
| infantis 35624 | PLUS | |
| (ALIGN ®) | ALIGN ® (1 capsule once | |
| | daily, QD) | |

Screening occurs at the time patients are diagnosed via CT with an acute attack of diverticulitis. Randomization occurs within 7 days after the diagnosis of acute diverticulitis has been confirmed by CT. Enrolled patients will initially receive either ASACOL® or placebo for 10 to 14 days (i.e., until Visit 2), when dietary supplementation with ALIGN® or placebo are added. All patients must have completed their antibiotic regimen for acute diverticulitis prior to starting dietary supplementation with ALIGN® or its placebo.

### Visits

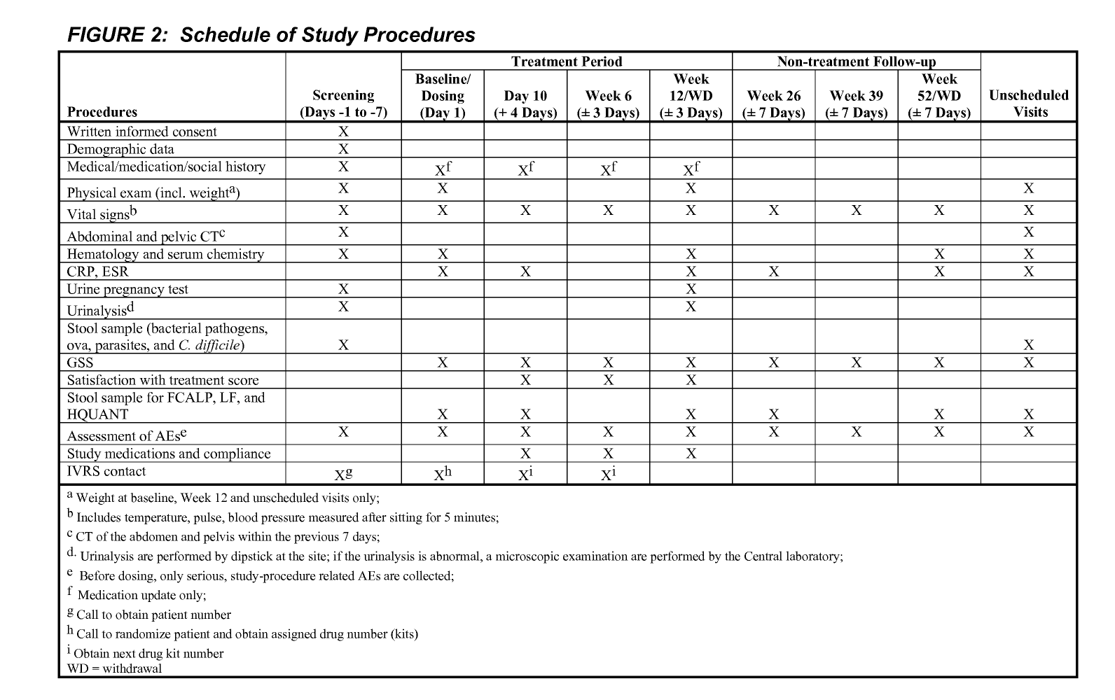

Visits occur at Baseline (Day 1) when patients will receive their first dose of study drug, Day 10 (+4) (Visit 2), Week 6±3 days (Visit 3), and Week 12±3 days (Visit 4) (FIG. 1). Patients are followed up for an additional 9 months (non-treatment period), with office visits at Week 26±7 days (Visit 5), Week 39±7 days (Visit 6), and Week 52±7 days (Visit 7). The schedule of events is displayed in tabular form in FIG. 2.

### Baseline Visit/Dosing (Day 1)

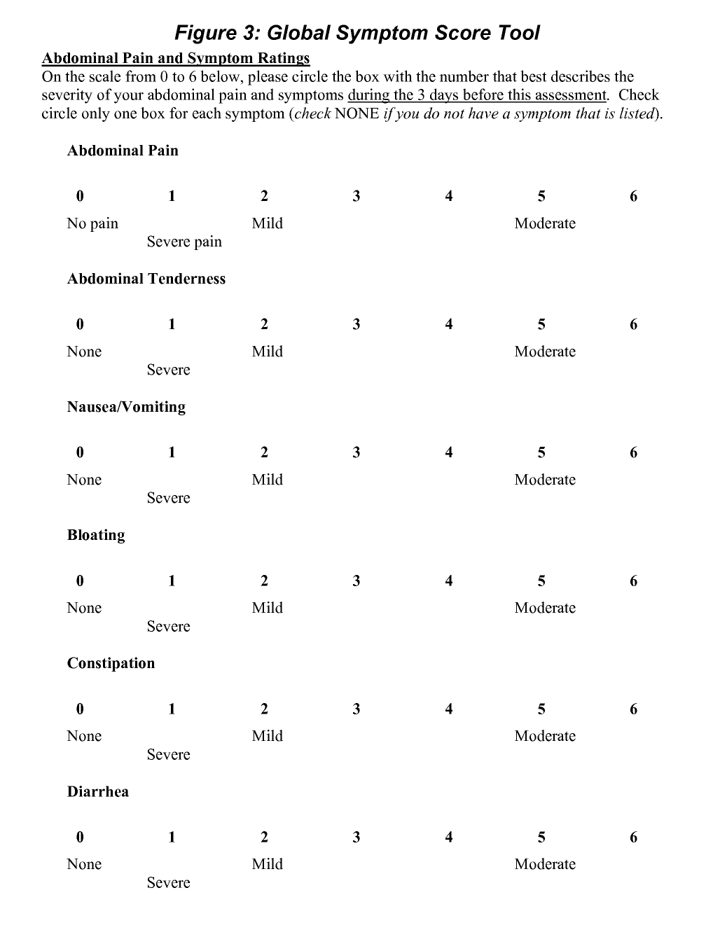

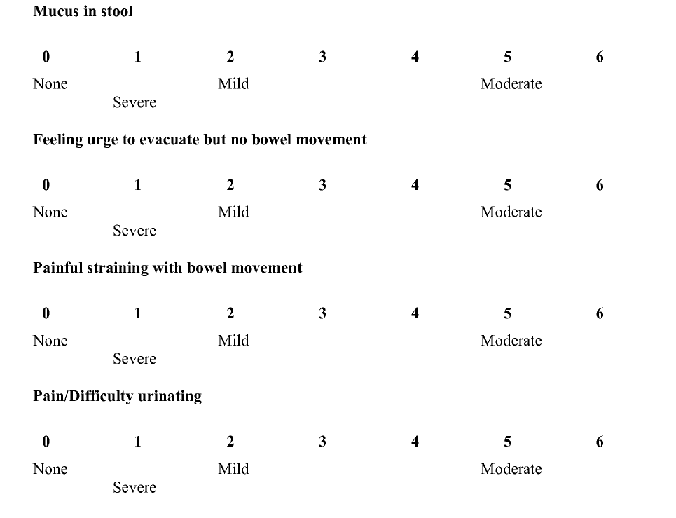

The baseline visit occurs within 7 days after the screening assessment. The following procedures are performed at this visit:
a. Medical and medication history update to include any changes or new medications since screening if applicable, and to ensure there is no change in medical condition that, in the Investigator's opinion, would interfere with study participation;
b. Physical examination update, including weight;
c. Recording of vital signs (temperature, pulse, blood pressure measured after sitting for 5 minutes);
d. Blood sample for hematology tests, C-reactive protein (CRP), erythrocyte sedimentation rate (ESR), and serum chemistry tests;
e. Gastrointestinal symptom severity assessment based on the patients' recall of the previous 3 days, using the Global Symptom Score (GSS) tool set forth at FIG. 3;
f. Stool sample for fecal calprotectin (FCALP), lactoferrin (LF), and HemoQuant (HQUANT);
g. Collection of serious, study procedure related, nontreatment-emergent AEs;
h. Obtain IVRS study kit number and dispense study medication;
i. Review dosing instructions with the patient and stress the importance of compliance;
j. Observe patient taking the first dose of study medication;
k. Schedule all study visits; all future visits must be scheduled from the first day (Day 1) of dosing with study medication. During the 12-week treatment period, future visits after Day 10 visit may deviate from the ideal date by ±3 days, including any necessary repeat procedures or rescheduled visits. During the non-treatment follow-up period, the Weeks 26, 39, and 52 visits may deviate from the ideal date by ±7 days;
l. Remind patient to bring all unused study medication, any empty blister cards to their next visit.

In addition, patients are given written dietary advice.

### Day 10 Visit (+4 Days)

The Day 10 visit may occur up to Day 14. The following procedures are performed at this visit:
a. Medication history updated to include any changes or new medications since baseline;
b. Recording of vital signs (temperature, pulse, blood pressure measured after sitting for 5 minutes);
c. Blood sample for CRP and ESR;
d. Gastrointestinal symptom severity assessment based on the patients' recall of the previous 3 days, using the Global Symptom Score (GSS) tool set forth at FIG. 3;
e. Stool sample for FCALP, LF, and HQUANT;
f. Assessment of AEs;
g. Counting of returned study medication and recording of number of returned tablets;
h. Start daily dietary supplementation with ALIGN® or placebo after cessation of antibiotic course.

### Week 6 Visit (±3 Days)

The following are performed at this visit:
a. Medication updated to include any changes or new medications since baseline;
b. Recording of vital signs (temperature, pulse, blood pressure measured after sitting for 5 minutes);
c. Gastrointestinal symptom severity assessment based on the patients' recall of the previous 3 days, using the Global Symptom Score (GSS) tool set forth at FIG. 3;
d. AE assessment;
e. Counting of returned study medication and recording of number of returned tablets;
f. Study medication dispensed via IVRS, dosing instructions reviewed with the patient, and importance of compliance stressed with the patient.

### Week 12 (±3 Days)

The following are performed at this visit:
a. Medication update to include any changes or new medications since last visit;
b. Physical examination including weight;
c. Recording of vital signs (temperature, pulse, blood pressure measured after sitting for 5 minutes);
d. Blood sample for hematology tests, CRP, ESR, and serum chemistry tests;
e. Urine pregnancy test, if applicable.
f. Urinalysis, using dipstick at the site.
g. Gastrointestinal symptom severity assessment based on the patients' recall of the previous 3 days, using the Global Symptom Score (GSS) tool set forth at FIG. 3;
h. Stool sample for FCALP, LF, and HQUANT;
i. AE assessment;
j. Counting of returned study medication and recording of number of returned tablets;
k. Instructions reviewed with the patient for the follow-up period of non-treatment (13 to 52 weeks). The importance of the 6-, 9-, and 12-month follow-up clinic visits are stressed with the patient, as well as the importance of contacting the Investigator if the GI symptoms worsen or if a recurrent attack of acute diverticulitis occurs during the non-treatment period.

### Week 26 (±7 Days)

The following are collected at this visit:
a. Recording of vital signs (temperature, pulse, blood pressure measured after sitting for 5 minutes);
b. Blood sample for CRP and ESR;
c. Gastrointestinal symptom severity assessment based on the patients' recall of the previous 3 days, using the Global Symptom Score (GSS) tool set forth at FIG. 3;
d. Stool sample for FCALP, LF, PMN-E, and HQUANT;
e. AE assessment.

### Week 39 (±7 Days)

The following are collected at this visit:
a. Recording of vital signs (temperature, pulse, blood pressure measured after sitting for 5 minutes);
b. Gastrointestinal symptom severity assessment based on the patients' recall of the previous 3 days, using the Global Symptom Score (GSS) tool set forth at FIG. 3;
c. AE assessment.

### Week 52 (±7 Days)

The following are collected at this visit:
a. Recording of vital signs (temperature, pulse, blood pressure measured after sitting for 5 minutes);
b. Blood sample for hematology tests, CRP, ESR, and serum chemistry tests;
c. Gastrointestinal symptom severity assessment based on the patients' recall of the previous 3 days, using the Global Symptom Score (GSS) tool set forth at FIG. 3;
d. Stool sample for FCALP, LF, and HQUANT;
e. AE assessment.

### Efficacy Assessments

The primary efficacy endpoint is the composite score of diverticulitis symptoms of interest. The symptoms of interest are listed in the GSS tool set forth at FIG. 3. The composite score at each visit (Baseline, Day 10, Week 6, and Week 12) is calculated as the sum of the 10 symptoms of interest. The primary efficacy endpoint is the change in the composite score from baseline at Week 12.

The secondary efficacy endpoints include:
1. The percentage of responders in each treatment group at Week 6, Week 12. A responder is defined as a patient whose scores for all symptoms of interest (set forth in the GSS tool at FIG. 3) are either 0 or 1;
2. The changes in GSS from Baseline at Day 10, Week 6, and Week 12;
3. The changes in GSS from Day 10 at Week 6 and Week 12;
4. For each of the GSS components, the percentage of patients who have a score of 0 or 1.
¹ Kang J Y, Hoare J, Tinto A, Subramanian S, Ellis C, Majeed A, et al. Diverticular disease of the colon—on the rise: a study of hospital admissions in England between 1989/1990 and 1999/2000. Aliment Pharmacol Ther. 2003; 17:1189-95.
² Warner E, Crighton E J, Moineddin R, Mamdani M, Upshur R. Fourteen-year study of hospital admissions for diverticular disease in Ontario. Can J. Gastroenterol. 2007; 21:97-9.
³ Simpson J, Scholefield J H, Spiller R C. Origin of symptoms in diverticular disease. Br J. Surg. 2003; 90:899-908.
⁴ Yamada T, Alpers D H, Laine L, Owyang C, Powell D W. In: Textbook of Gastroenterology. 3rd ed. Philadelphia: Lippincott, Williams, and Wilkins; 1999: pp 1929.
⁵ Petruzziello L, Tacopini F, Bulajic M, Shah S, Costamagna G. Review article: uncomplicated diverticular disease of the colon. Aliment Parmacol Ther. 2006; 23:1379-91.
⁶ Sandler R S, Everhart J E, Donowitz M, Adams E, Cronin K, Goodman C, et al. The burden of selected digestive diseases in the United States. Gastroenterology 2002; 122:1500-11.
⁷ Ambrosetti P, Becker C, Terrier F. Colonic diverticulitis: impact of imaging on surgical management—a prospective study of 542 patients. Eur Radiol. 2002; 12:1145-9.
⁸ Ambrosetti P, Jenny A, Becker C, Terrier T F, Morel P. Acute left colonic diverticulitis—compared performance of computed tomography and water-soluble contrast enema: prospective evaluation of 420 patients. Dis Colon Rectum. 2000; 43:1363-7.
⁹ Chautems R C, Ambrosetti P, Ludwig A, Mermillod B, Morel P, Soravia C. Long-term follow-up after first acute episode of sigmoid diverticulitis: is surgery mandatory?: a prospective study of 118 patients. Dis Colon Rectum. 2002; 45:962-6.
¹⁰ Richards R J, Hammitt J K. Timing of prophylactic surgery in prevention of diverticulitis recurrence: a cost-effectiveness analysis. Dig Dis Sci. 2002; 47:1903-8.
¹¹ Breen R E, Corman M L, Robertson W G, Prager E D. Are we really operating on diverticulitis? Dis Colon Rectum. 1986; 29: 174-6.
¹² Salem L, Veenstra D L, Sullivan S D, Flum D R. The timing of elective colectomy in diverticulitis: a decision analysis. J Am Coll Surg. 2004; 199:904-12.
¹³ Aydin H N, Remzi F H. Diverticulitis: when and how to operate? Dig Liver Dis. 2004; 36:435-45.
¹⁴ Wong W D, Wexner S D, Lowry A, Vernava A 3^{rd}, Burnstein M, Denstman F, et al. Practice parameters for the treatment of sigmoid diverticulitis-supporting documentation. The Standards Task Force. The American Society of Colon and Rectal Surgeons. Dis Colon Rectum. 2000; 43:290-7.
¹⁵ Broderick-VIIIa G, Burchette R J, Collins J C, Abbas M A, Haigh P I. Hospitalization for acute diverticulitis does not mandate routine elective colectomy. Arch Surg. 2005; 140:576-81.
¹⁶ Guzzo J, Hyman N. Diverticulitis in young patients: is resection after a single attack always warranted? Dis Colon Rectum. 2004; 47:1187-90.
¹⁷ Horgan A F, McConnell E J, Wolff B G, The S, Paterson C. Atypical diverticular disease: surgical results. Dis Colon Rectum. 2001; 44:1315-8.
¹⁸ Janes S, Meagher A, Frizelle F A. Elective surgery after acute diverticulitis. Br J. Surg. 2005; 92:133-42.
¹⁹ Kaiser A M, Jiang J K, Lake J P, Ault G, Artinyan A, Gonzalez-Ruiz C, et al. The management of complicated diverticulitis and the role of computed tomography. Am J. Gastroenterol. 2005; 100:910-7.
²⁰ Parks T G. Natural history of diverticular disease of the colon. A review of 521 cases. Br J. Surg. 1969; 4:639-42.
²¹ Parks T G, Connell A M. The outcome in 455 patients admitted for treatment of diverticular disease of the colon. Br J. Surg. 1970; 57:775-8.
²² Munson K D, Hensien M A, Jacob L N, Robinson A M, Liston W A. Diverticulitis. A comprehensive follow-up. Dis Colon Rectum. 1996; 39:318-22.
²³ Narayan R, Floch M H. Microscopic colitis as part of the natural history of diverticular disease (Abstract). Am J Gastroenterol 2002; 97(Suppl): 112.
²⁴ [No authors listed] Position of the American Dietetic Association: health implications of dietary fiber. J Am Diet Assoc. 1997; 97:1157-9.
²⁵ Gear J S, Ware A, Fursdon P, Mann J I, Nolan D J, Brodribb A J, et al. Symptomless diverticular disease and intake of dietary fibre. Lancet. 1979; 1:511-4.
²⁶ Simpson J, Spiller R. Colonic diverticular disease. Clin Evid. 2005; (14):543-50.
²⁷ Simpson J, Scholefield J H, Spiller R C. Pathogenesis of colonic diverticula. Br J. Surg. 2002; 89:546-54.
²⁸ Gionchetti P, Amadini C, Rizzello F, Venturi A, Palmonari V, Morselli C, et al. Probiotics—role in inflammatory bowel disease. Dig Liver Dis. 2002; 34(Suppl 2):S58-62.
²⁹ Hopkins M J, Sharp R, Macfarlane G T. Variation in human intestinal microbiota with age. Dig Liver Dis. 2002; 34(Suppl 2):S12-8.
³⁰ Saunier K, Dore J. Gastrointestinal tract and the elderly: functional foods, gut microflora and healthy ageing. Dig Liver Dis. 2002; 34(Suppl 2)S19-24.
³¹ Floch M H, Fuchs H M. Modification of stool content by increased bran intake. Am J Clin Nutr. 1978; 31(10 Suppl):S185-9.

What is claimed is:

1. A method of treating a condition selected from diverticular disease, diverticulitis, and combinations thereof, the method comprising administering to a mammal in need of such treatment a composition comprising a Bifidobacterium spp.

2. The method according to claim 1 wherein the Bifidobacterium spp. comprises bacteria selected from the group consisting of Bifidobacterium longum, Bifidobacterium infantis, Bifidobacterium bifidum, and mixtures thereof.

3. The method according to claim 2 wherein the Bifidobacterium spp. comprises Bifidobacterium infantis.

4. The method according to claim 2 wherein a single dose of the composition comprises at least about 10⁸ cfu of the Bifidobacterium spp.

5. The method according to claim 4 wherein a single dose of the composition comprises from about 10⁹ cfu to about 10¹² cfu of the Bifidobacterium spp.

6. The method according to claim 5 wherein the Bifidobacterium spp. comprises Bifidobacterium infantis.

7. The method according to claim 6 wherein administration of the composition is oral.

8. The method according to claim 7 wherein administration of the composition is at least once daily.

9. The method according to claim 2 wherein administration of the composition is selected from the group consisting of oral, rectal, intramuscular, subcutaneous, intravenous, transdermal, and combinations thereof.

10. The method according to claim 8 wherein administration of the composition is selected from the group consisting of oral, rectal, and combinations thereof.

11. The method according to claim 1 further comprising administering to the mammal an anti-inflammatory.

12. The method according to claim 11 wherein the anti-inflammatory is selected from the group consisting of salicylates, arylalkanoic acids, 2-arylpropionic acids, N-arylanthranillic acids, pyrazolidines, oxicams, coxibs, sulphonanilides, licofelone, omega fatty acids, and combinations thereof.

13. The method according to claim 12 wherein the anti-inflammatory is a 5-aminosalicylic acid.

14. The method according to claim 11 wherein administration of the Bifidobacterium spp. and the anti-inflammatory is contemporaneous.

15. The method according to claim 14 wherein the composition comprising the Bifidobacterium spp. also comprises the anti-inflammatory.

16. The method according to claim 11 wherein administration of the anti-inflammatory is prior to the administration of the Bifidobacterium spp.

17. The method according to claim 11 administration of the Bifidobacterium spp. is prior to the administration of the anti-inflammatory.

18. The method according to claim 11 further comprising administering to the mammal an antibiotic.

19. The method according to claim 18 wherein the antibioitic is selected from the group consisting of metronidazole, cephalexin, ciprofloxacin, levofloxacin, moxifloxacin, gemifloxacin, balofloxacin, gatifloxacin, grepafloxacin, pazufloxacin, sparfloxacin, temafloxacin, enoxacin, fleroxacin, lomefloxacin, nadifloxacin, norfloxacin, ofloxacin, pefloxacin, rufloxacin, trovofloxacin, tosufloxacin, clindamycin, tetracycline, chloramphenicol, cefoxitin, cefmetazole, cefotetan, doxycycline, erythromycin, imipenem, meropenem, ticarcillin, pipercillin, mezocillin, tazobactam, ampicillin, nitrofurantion, amoxicillin, trimethoprim, sulfamethoxazole, rifampin, penicillin, penicillin g, gentamicin, vancomycin, cefotaxime, ceftriazone, naldixic cinoxacin, amifloxacin and combinations thereof.

20. The method according to claim 19 wherein administration of the antibiotic is prior to the administration of the Bifidobacterium spp. and prior to the administration of the anti-inflammatory.

21. A method of treating a condition selected from diverticular disease, diverticulitis, and combinations thereof, the method comprising administering to a mammal in need of such treatment a probiotic, an anti-inflammatory, and an antibiotic.

22. The method according to claim 21 wherein the administration of the antibiotic is prior to the administration of the probiotic and prior to the administration of the anti-inflammatory.

23. The method according to claim 22 comprising administration of a composition, wherein the composition comprises the probiotic and the anti-inflammatory.

24. The method according to claim 22 wherein the anti-inflammatory is selected from the group consisting of salicylates, arylalkanoic acids, 2-arylpropionic acids, N-arylanthranillic acids, pyrazolidines, oxicams, coxibs, sulphonanilides, licofelone, omega fatty acids, and combinations thereof.

25. The method according to claim 24 wherein the anti-inflammatory is a 5-aminosalicylic acid.

26. The method according to claim 25 wherein the antibioitic is selected from the group consisting of metronidazole, cephalexin, ciprofloxacin, levofloxacin, moxifloxacin, gemifloxacin, balofloxacin, gatifloxacin, grepafloxacin, pazufloxacin, sparfloxacin, temafloxacin, enoxacin, fleroxacin, lomefloxacin, nadifloxacin, norfloxacin, ofloxacin, pefloxacin, rufloxacin, trovofloxacin, tosufloxacin, clindamycin, tetracycline, chloramphenicol, cefoxitin, cefmetazole, cefotetan, doxycycline, erythromycin, imipenem, meropenem, ticarcillin, pipercillin, mezocillin, tazobactam, ampicillin, nitrofurantion, amoxicillin, trimethoprim, sulfamethoxazole, rifampin, penicillin, penicillin g, gentamicin, vancomycin, cefotaxime, ceftriazone, naldixic cinoxacin, amifloxacin and combinations thereof.

27. A kit comprising:
a) a probiotic; and
b) an anti-inflammatory.

28. The kit according to claim 27 wherein the anti-inflammatory is selected from the group consisting of salicylates, arylalkanoic acids, 2-arylpropionic acids, N-arylanthranillic acids, pyrazolidines, oxicams, coxibs, sulphonanilides, licofelone, omega fatty acids, and combinations thereof.

29. The kit according to claim 28 wherein the anti-inflammatory is a 5-aminosalicylic acid.

30. The kit according to claim 29 wherein the probiotic is a Bifidobacterium spp.

31. The kit according to claim 29 comprising a first composition, wherein the first composition comprises from about 1 mg to about 1000 mg of the 5-aminosalicylic acid.

32. The kit according to claim 31 comprising a second composition, wherein the second composition comprises the Bifidobacterium spp.

33. The kit according to claim 32 wherein the Bifidobacterium spp. comprises bacteria selected from the group consisting of Bifidobacterium longum, Bifidobacterium infantis, Bifidobacterium bifidum, and mixtures thereof.

34. The kit according to claim 33 wherein the second composition comprises at least about 10⁸ cfu of the Bifidobacterium spp.

35. The kit according to claim 34 wherein the second composition comprises from about 10⁹ cfu to about cfu 10¹² of the Bifidobacterium spp.

36. The kit according to claim 35 wherein the Bifidobacterium spp. is Bifidobacterium infantis.

37. The kit according to claim 32 wherein the Bifidobacterium spp. is Bifidobacterium infantis.

38. The kit according to claim 27, further comprising an antibiotic.

39. The kit according to claim 38 wherein the antibioitic is selected from the group consisting of metronidazole, cephalexin, ciprofloxacin, levofloxacin, moxifloxacin, gemifloxacin, balofloxacin, gatifloxacin, grepafloxacin, pazufloxacin, sparfloxacin, temafloxacin, enoxacin, fleroxacin, lomefloxacin, nadifloxacin, norfloxacin, ofloxacin, pefloxacin, rufloxacin, trovofloxacin, tosufloxacin, clindamycin, tetracycline, chloramphenicol, cefoxitin, cefmetazole, cefotetan, doxycycline, erythromycin, imipenem, meropenem, ticarcillin, pipercillin, mezocillin, tazobactam, ampicillin, nitrofurantion, amoxicillin, trimethoprim, sulfamethoxazole, rifampin, penicillin, penicillin g, gentamicin, vancomycin, cefotaxime, ceftriazone, naldixic cinoxacin, amifloxacin and combinations thereof.
